(19) United States
(12) Patent Application Publication
(10) Pub. No.: US 2023/0149832 A1
(43) Pub. Date: May 18, 2023

(54) SURFACE FUNCTIONALIZED AFFINITY MEMBRANES

(71) Applicant: NANOPAREIL, LLC, Dakota Dunes, SD (US)

(72) Inventors: Todd J. Menkhaus, Dakota Dunes, SD (US); Steven Schneiderman, Dakota Dunes, SD (US)

(21) Appl. No.: 17/937,251

(22) Filed: Sep. 30, 2022

Related U.S. Application Data
(63) Continuation of application No. PCT/US2021/025438, filed on Apr. 1, 2021.
(60) Provisional application No. 63/003,629, filed on Apr. 1, 2020.

(51) Int. Cl. B01D 15/38; B01J 20/28; G01N 33/543; B01D 69/02; B01D 69/10; B01D 69/14; B01D 67/00; B01J 20/32

(52) U.S. Cl. CPC B01D 15/3828; B01J 20/28038; G01N 33/54353; B01D 69/02; B01D 69/1071; B01D 69/144; B01D 67/0002; B01D 67/00931; B01J 20/3255; B01D 2325/12; B01D 2325/36; B01D 2325/42; B01D 2325/38; B01J 20/3274; B01D 2323/36; B01J 2220/80; B01J 2220/54

(57) ABSTRACT

The present disclosure provides surface functionalized affinity membranes. The surface functionalized affinity membranes can provide increased binding capacity through improved coupling chemistries, ligand densities, spacer arm types, and spacer arm lengths. Methods of preparing the surface functionalized affinity membranes and methods of using the surface functionalized affinity membranes to isolate targets of interest, including nucleic acid molecules and proteins, from a sample are also provided.

### CROSS REFERENCE TO RELATED APPLICATIONS

This application is a by-pass Continuation of PCT/US2021/025438, filed Apr. 1, 2021, which claims priority to provisional application U.S. Ser. No. 63/003,629, filed Apr. 1, 2020, which are incorporated herein by reference in their entireties.

### GRANT REFERENCE

This invention was made with government support under Grant No. IIP-1329377, awarded by the National Science Foundation. The Government has certain rights in this invention.

### TECHNICAL FIELD

This disclosure relates generally to the field of chromatography. More specifically, the disclosure relates to modifications to the surface of membranes for tailored arrangement and immobilization of affinity ligands.

### BACKGROUND

The immense potential of monoclonal antibodies as therapeutic agents and diagnostic tools has led to significant research efforts in the biotechnology industry to produce high purity antibodies. Large-scale production of immunoglobulin G (IgG) requires both upstream processing units that can handle high volumes and efficient downstream processing units to produce extremely pure products. The recent improvements achieved in the upstream cell culture titers of antibody production in the last decade put tremendous pressure on the downstream processing, which can account for up to 80% of the actual production cost of the IgG. Protein A affinity chromatography is typically used as a first step in the purification of immunoglobulins. The high affinity interaction between the Fc region of the IgG and Protein A forms the basis of the affinity purification of many subclasses of IgG. Because of this reversible interaction, passage of cell culture fluid (CCF) through a Protein A column leads to the binding of IgG's and the flow of unwanted impurities such as viruses, host cell proteins, DNA, etc. through the chromatography media. However, traditional resin-based Protein A chromatography suffers from limitations such as high pressure drop, elevated buffer consumption, low capacity, low productivity (especially at high flow rates), high cost, etc. Thus, significant research efforts have been focused on the development of Protein A-based membrane adsorbers as an alternative to resin-based purification step in IgG production. Several macroporous membranes have been reported. Although these membranes have faster adsorption kinetics and high permeability compared to packed bed resins, they suffer from low binding capacity because of the limited surface area available for adsorption.

Thus, there exists a need for alternative compositions and methods for purification of immunoglobulins and other biomolecules, which provide favorable adsorption kinetics, high surface area, high porosity, high binding capacity and ease of functionalization.

### SUMMARY OF PREFERRED EMBODIMENTS

In a preferred embodiment, the surface functionalized affinity membranes comprise a membrane support, a plurality of spacer arms immobilized on the membrane support, each spacer arm comprising a terminus, and a ligand coupled to the terminus. The membrane support can comprise a cast membrane, an electrospun membrane, or a combination thereof. In a preferred embodiment, the membrane support further comprises one or more inert functional groups immobilized on the surface of the membrane support.

In a preferred embodiment, a method of preparing a surface functionalized affinity membrane comprises providing a nanofiber membrane support; immobilizing a spacer arm on the membrane support, wherein each spacer arm comprises a terminus; and coupling a ligand to the terminus of the spacer arm via a reactive functional group. The membrane support can comprise a cast membrane, an electrospun membrane, or a combination thereof. In a preferred embodiment, the membrane support further comprises one or more inert functional groups immobilized on the surface of the membrane support.

In a preferred embodiment, a method of isolating a target of interest from a sample comprises contacting the sample comprising the target of interest with the surface functionalized affinity membrane to adsorb the target of interest. The membrane support can comprise a cast membrane, an electrospun membrane, or a combination thereof. In a preferred embodiment, the membrane support further comprises one or more inert functional groups immobilized on the surface of the membrane support.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The following drawings form part of the specification and are included to further demonstrate certain embodiments or various aspects of the invention. In some instances, embodiments of the invention can be best understood by referring to the accompanying figures in combination with the detailed description presented herein. The description and accompanying figures may highlight a certain specific example, or a certain aspect of the invention. However, one skilled in the art will understand that portions of the example or aspect may be used in combination with other examples or aspects of the invention.

FIG. 1 shows various functional groups capable of coupling amine-containing molecules. This includes epoxides or oxiranes, NHS esters, aldehydes, and sulfonyl chlorides (from left to right).

FIG. 2A shows the distinction between activation density and ligand density for the immobilization of Protein A onto an epoxide activated surface.

FIG. 2B shows a surface that has surpassed minimum ligand density due to steric interactions and size limitations preventing an increase in IgG binding capacity.

FIG. 2C shows a surface optimized by changing the activation density and tailoring the surface functionalization to include inert functional groups and an exemplary affinity ligand (Protein A) to maximize the ligand efficiency.

FIG. 2D shows a surface with multiple different spacer molecule lengths to maximize the binding of IgG.

FIG. 3 shows introduction of a hydrocarbon spacer molecule for subsequent activation with a carboxylic acid group. Where X=F, Cl, Br, or I. n=0-infinity.

FIG. 4 shows various bis-epoxide spacer/linker molecules immobilized onto a cellulose matrix. This includes butanediol diglycidal ether, Poly(ethylene glycol) diglycidyl ether (average Mn=500), and Poly(ethylene glycol) diglycidyl ether (average Mₙ=2000) (from left to right).

FIG. 5 demonstrates a possible optimized surface with varied epoxide, and therefore ligand, density as well as different spacer molecule lengths to maximize potential IgG binding.

FIG. 6 shows the effect of varying spacer molecule length on protein A to IgG bind usefulness for epoxide surfaces.

FIG. 7 shows the effect of varying spacer molecule length on IgG binding capacity per ml of membrane.

FIG. 8 shows the effect of varying spacer molecule length on protein uptake from 50 mg protein A in 1 ml.

FIG. 9 shows the effect of varying spacer molecule length on protein A to IgG bind usefulness for NHS surfaces.

FIG. 10 shows the effect of varying spacer molecule length on IgG binding capacity per ml of membrane.

### DETAILED DESCRIPTION

The present disclosure relates to surface functionalized affinity membranes having a plurality of ligands attached to the surface of the membrane via spacer arms. The affinity membranes have many advantages over existing separation technologies including packed bed resins, macroporous membranes, and surface functionalized membranes. For example, the affinity membranes can have higher binding capacity due to high specific surface area, increased ligand density and increased ligand efficiency. According to the present disclosure, these advantages can be achieved by tailored coupling chemistries, ligand densities, spacer arm types, and spacer arm lengths. The embodiments described herein can be applied to any affinity membranes which can vary and are understood by skilled artisans.

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

The term “about,” as used herein, refers to variation in the numerical quantity that can occur, for example, through typical measuring techniques and equipment, with respect to any quantifiable variable, including, but not limited to, mass, volume, time, temperature, length, molecular weight, density, absorption, and binding capacity. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The terms “membrane”, “felt”, and “mat” as used herein are interchangeable and refer to a non-woven or randomly overlaid collection of fibers, optionally formed by the application of additional steps to form the final composition, including but not limited to annealing, fusing, heating, pressing, coating, and crosslinking. The fibers forming a membrane can be of any suitable diameter. In a preferred embodiment, the membranes comprise nanofibers, microfibers, or a combination of nanofibers and microfibers. Membranes can be cylindrical or substantially planar.

The term “membrane support” refers to a membrane, including, cast membranes, electrospun membranes, or a combination of cast and electrospun membranes. Membrane support can comprise multiple membranes can be layered together.

The term “nanofiber membrane” as used herein refers to a collection of nanofibers, which may also include microfibers added for strength, enhancing flux, and other properties.

The term “microfibers” as used herein refers to fibers with diameters larger than 1.0 micrometer, and generally between 1.0 micrometer and 1.0 millimeter.

The term “nanofibers” as used herein refers to fibers with diameters smaller than of 1.0 micrometer, and generally between 10 nanometers and 1.0 micrometer, such as between 200 nm and 600 nm.

The term “hybrid nanofiber membrane” as used herein refers to a non-woven or randomly overlaid collection of fibers consisting of at least two types of polymers in a combination of single component fibers or composite fibers with either at least one other single component fiber or at least one other composite fiber.

The term “composite nanofibers” as used herein are nanofibers produced from at least two different polymers.

The term “electrospinning” as used herein refers to the application of electric forces to the spin dope to form nanofibers.

The term “spin dope” as used herein refers to the polymer solution that is used in the electrospinning process.

The term “capacity” as used herein refers to the amount of product bound per unit of adsorbent.

The term “affinity chromatography” is used herein for the specific mode of chromatography where the ligand interacts with target via biological affinity in a lock-key fashion. Examples of useful interactions in affinity chromatography are e.g. enzyme-substrate interaction, biotin-avidin interaction, antibody-antigen interaction, etc.

The terms “protein”, “polypeptide”, or “peptide” can be used interchangeably and refer to any natural or recombinant molecule comprising amino acids joined together by peptide bonds between adjacent amino acid residues. A “peptide bond”, “peptide link”, or “amide bond” is a covalent bond formed between two amino acids when the carboxyl group of one amino acid reacts with the amino group of the other amino acid. The terms “amino acid”, “amino acid residue”, and “residue” may be used interchangeably herein.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

A “virus-like particle” or “VLP” as used herein refers to at least one virus particle, which does not contain any nucleic acid. VLPs can thus be used for vaccination or for inducing an immunogenic reaction in a subject. However, due to the absence of nucleic acids, VLPs will not be able to replicate in a host cell and are thus non-replicative.

The term “lipid nanoparticle” or “LNP” as used herein is a general term to describe lipid-based particles in the submicron range. LNPs can have structural characteristics of liposomes and/or have alternative non-bilayer types of structures. LNPs constitute an alternative to other particulate systems, such as emulsions, liposomes, micelles, microparticles and/or polymeric nanoparticles, for the delivery of active ingredients, such as oligonucleotides and small molecule pharmaceuticals.

The term “exosomes” as used herein refers to small, secreted vesicles (typically about 30-150 nm) which may contain, or have present in their membrane, nucleic acid, protein, or other biomolecules and may serve as carriers of this cargo between diverse locations in a body or biological system.

The terms “purifying,” “separating,” or “isolating,” as used interchangeably herein, refer to increasing the degree of purity of a molecule of interest or a target molecule from a composition or sample comprising the molecule and one or more impurities.

The terms “spacer molecule,” “spacer arm” or “linker” as used interchangeably herein refer to an element that distances a ligand from the membrane support.

The term “ligand” as used herein refers to molecules or compounds capable of interaction with target compounds, such as antibodies.

As used herein, the term “sample” refers to any liquid or solid material to be used in the processes described herein. For example, a sample can be a solution containing eukaryotic or prokaryotic cells or cellular material, or virus or viral material, or bacteria or bacterial material, or microorganisms or pathogens. A sample can be essentially water, or a buffered solution or be composed of any artificially introduced chemicals and may or may not contain nucleic acids or proteins.

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples can include biological solid material or biological fluid or a biological tissue. Examples of biological solid materials include tumors, cell pellets, or biopsies. Examples of biological fluids include cell cultures, cell homogenates, suspension of cells in a medium, urine, blood, plasma, serum, sweat, saliva, semen, stool, sputum, cerebral spinal fluid, mouth wash, tears, mucus, sperm, amniotic fluid, or the like. Biological tissues are aggregates of cells, usually of a particular kind, together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries, and individual cell(s). Also included the definition of biological sample are soil, water and other environmental samples including industrial waste and natural bodies of water (lakes, streams, rivers, oceans) that can contain viruses, bacteria, fungi, algae, protozoa, and components thereof.

### Membrane Support

The affinity membrane compositions of the present disclosure include a membrane support. The membrane support can be a cast membrane, electrospun membrane, or a combination thereof. The membrane support is comprised of fibers. The fibers forming the membrane support can be of any desired diameter. In a preferred embodiment, the membrane support comprises nanofibers, microfibers, or a mixture thereof. The membrane support can be made of various types of polymeric materials. In a preferred embodiment the membrane support comprises cellulose, a cellulose derivative, a non-cellulose polymer, a composite nanofiber, or a mixture thereof.

In a preferred embodiment, the membrane comprises cellulose. Cellulose is a preferred base material for the affinity membrane compositions because of its very low non-specific binding characteristics allowing for the ability to interact specifically with the targeted biological molecules through the intended immobilized ligand and not directly with the base material. The hydroxyl (—OH) groups in the cellulose can easily be converted to other desired functionality for coupling reactions using various surface grafting methods.

The membrane support can be prepared or obtained in prepared form. The membrane support can be a hybrid membrane containing cellulose and a non-cellulose-based polymer. Suitable hybrid membranes and methods of preparing the same are described in U.S. Patent Publication No. 2015/0360158, which is incorporated herein in its entirety. If the membrane support is a hybrid membrane, preferably the hybrid membrane comprises at least 30 wt. %, at least 40 wt. %, at least 50 wt. %, at least 60 wt. %, or at least 70 wt. % cellulose-based fibers. In another embodiment, the membrane support can consist of or consist essentially of cellulose-based fibers. The membrane support can comprise a surface functionalization and can be further surface functionalized according to the methods described herein.

In a preferred embodiment, the majority by mass in a hybrid membrane can be from cellulose-based polymers, incorporation of additional types of fibers within the membranes can provide functionality desired for applications of the membranes. Accordingly, it is desirable to have additional fibers within the membranes because they can provide increased mechanical strength to the membrane, allow for multiple functionalities to be incorporated into the membrane, and provide stability to the manufacturing process.

Many non-cellulose polymers have been successfully used in forming membrane supports by casting or electrospinning. Non-cellulose polymer suitable for forming a membrane support include, but are not limited to, (1) thermoplastic homopolymers such as vinyl polymers, acrylic polymers, polyamides, polyesters, polyethers, and polycarbonates, (2) thermoplastic copolymers such as vinyl-co-vinyl polymers, acrylic-co-acrylic copolymers and vinyl-coacrylic polymers, (3) elastomeric polymers such as triblock copolymer elastomers, polyurethane elastomers, and ethylene-propylene-diene-elastomers, (4) high performance polymers such as polyimides and aromatic polyamides, (5) liquid crystalline polymers such as poly(p-phenylene terephthalamide) and polyaramid, (6) textile polymers such as polyethylene terephthalate and polyacrylonitrile, (7) electrically conductive polymers such as polyaniline, as well as (8) biocompatible polymers (i.e. “biopolymers”) like polycaprolactone, polylactide, chitosan and polyglycolide. As described, the fibers forming a membrane support can comprise one or more of these polymers, may also be a copolymer of two or more of the above-named polymer species, and may be a composite fiber comprised of cellulose and/or a cellulose derivative as well as one of the foregoing polymers.

An exemplary electrospinning process can generally be described as follows:

Step 1: A spin dope (e.g., a polymer solution) is placed in a container with spinneret (1), and DC high voltage (2), usually in the range from 5-40 kilovolts, is applied to the solution through an electrode (e.g., a copper wire) (3). An electrically grounded collector (4) is placed at a certain distance (known as the gap distance) (5) away from the spinneret. The gap distance may range from a few centimeters up to one meter. When the electrostatic field reaches a critical value, and the electric force overcomes surface tension and viscoelastic forces, a jet/filament is ejected and travels straight for a certain distance (known as the jet length).

Step 2: The jet then starts to bend, forming helical loops. This phenomenon is termed “bending (or whipping) instability.” Typically, the bending instability causes the length of a jet to elongate by more than 10,000 times in a very short time period (50 ms or less). Thus, the elongational rate during the bending instability is extremely high (up to 1,000,000 s⁻¹). This extremely fast elongational rate can effectively stretch the chain of macromolecules and closely align them along the nanofiber axis.

Step 3: The jet solidifies, either though evaporation of the solvent or when the melt cools below the solid-liquid transition temperature. The longer the solidification time, the more the jet can be lengthened. The solidification time is related to many factors such as solvent vapor pressure, solvent diffusivity, volumetric charge density carried by the jet, and strength of the applied electrostatic field.

Optional Post-Electrospinning Processing

After solidification of the collected nanofibers, there are certain additional steps that can be performed in order to “customize” the nanofibers for particular uses. Exemplary additional steps are discussed below:

a. Heating and Pressing

In a preferred embodiment, membranes prepared from electrospun fibers can be heated and pressed together to anneal and/or fuse the fibers together.

b. Removal of a Polymer

In some cases, one or more of the polymers, and in particular a composite nanofiber comprising differentially removable polymers, one of the differentially removable polymers can be removed using elevated heat and/or solvent(s). Removal of a differentially removable polymer provides additional surface area and improved porosity of the remaining polymeric fibers. This is because after removal of the differentially removable polymer, the remaining polymer has controlled-sized “pores” left behind where the removed polymer used to occupy space. This additional “void space” provides greater surface area on the resultant membrane support that can, for example, increase adsorptive binding capacity for separations, improve selectivity of size-based separations, and improve throughput from additional porosity.

c. Cellulose Regeneration

In an embodiment employing derivatized cellulose, after preparation of the “as electrospun” nanofibers, the derivatized cellulose can be converted into cellulose through the process of regeneration. The regenerated cellulose will have the same properties as pure native cellulose described previously. The regeneration process is completed by contacting nanofibers containing derivatized cellulose with, for example, a strong base (e.g., sodium hydroxide), or other solvent. Following the regeneration reaction for conversion to cellulose, the nanofibers can be washed to remove any excess solvent used during the process.

More detailed electrospinning techniques are further disclosed in U.S. Pat. No. 9,604,168, which is incorporated herein in its entirety.

### Surface Functionalization

After preparing or obtaining the membrane support, the surface of the membrane support is functionalized. More specifically, the fiber surfaces are functionalized. The fiber surfaces can be functionalized regardless of their diameter. In a preferred embodiment, the fiber surfaces which are functionalized comprise nanofibers, microfibers, or a mixture thereof.

Preferably functionalization includes the addition of affinity ligands including, but not limited to, virus conjugates, antibodies, enzyme substrates, and small molecule biomimetics. Affinity ligands added may also include monoclonal antibodies, polyclonal antibodies, antibody fragments, nucleic acids, oligonucleotides, proteins, oligopeptides, polysaccharides, oligosaccharides, sugars, peptides, antigens, aptamers, affimers, small organic compounds, drugs and other ligands. Examples of suitable affinity ligands are available in the published literature and are well known. Preferably the ligand can bind an immunoglobulin. Non-limiting, examples of such ligands include Protein A, Protein G, and Protein L.

For use in bioseparation, the surface functionalized membranes are ideally biologically inert, meaning that they should resist non-specific binding of insoluble solids such as cells and cellular debris, as well as unwanted interactions with proteins, sugars, nucleic acids, viruses, and other soluble components present in many biologically produced systems.

In a preferred embodiment, surface functionalized membranes for use in bioseparation exhibit one or more of the following technical features: (1) small diameter fibers to allow for the largest amount of specific area; (2) well-controlled and narrow pore size distribution between fibers to allow for even flow distribution during adsorptive applications; (3) fibers having excellent mechanical and chemical stability to withstand potentially high operating pressures and harsh cleaning conditions; and (4) fibers having a well-defined and spatially consistent size and chemical composition. In a more preferred embodiment, the membrane support comprises crosslinked fibers. In a most preferred embodiment, the membrane support comprises crosslinked cellulose nanofibers; such as the crosslinked nanofibers described in U.S. Pat. No. 10,919,986, which is incorporated by reference in its entirety.

For adsorption processes, where macromolecular products such as proteins, nucleic acids, and viruses are the predominant targets, the large specific surface area associated with the surface functionalized membranes described herein provide a plurality of potential binding sites for adsorptive bioseparations. The membrane support can be modified by the addition of surface functionalizations to contain a plurality of binding sites and adsorption can occur on the surface of the fibers, which makes the binding sites immediately available without requiring the target molecule to diffuse internally. Each of the binding sites comprises an affinity ligand attached via a surface functionalization. In this respect, the affinity ligands are immobilized on the membrane support. Internal diffusion can often limit the capacity for many adsorption processes of bioproducts when using traditional porous resin beads because of the relatively large size of the target molecules. In addition, because the membrane support can be made from many different polymer- and cellulose-based fibers, the adsorption ligands can be tailored to meet the needs of a particular separation.

The surface functionalized affinity membranes described herein provide improvements over traditional resin bead separation techniques. Two of those improvements are that, (1) flow is through micro- and macro-pores of the membrane support (as opposed to tightly packed resin beads), and (2) that adsorption takes place on the surface of the fibers, where no internal diffusion is required. These factors reduce concerns of high-pressure drops with elevated flow rates, and eliminate the slow intra-particle diffusion required for adsorption within resin beads. We have found that the binding capacity of biomolecules to surface functionalized affinity membranes is similar in magnitude to resin beads, but can operate at processing flow rates over 10 times faster than packed beds. These factors allow for much faster processing times and potentially higher binding levels for purifying valuable biological products. This is highly desirable, especially for large biomolecules (molecular weights greater than 100 kDa, and/or hydrodynamic diameters of 20-300 nm), because they are difficult to purify using packed beds due to the mass transfer limitations within the small pores of resin beads.

### Coupling Chemistries

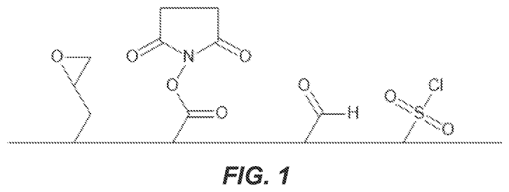

Various types of chemistry can be utilized to permanently bind the desired ligand to the surface of the membrane support. Choice of reaction chemistry must be selected such that no interfering side reactions under the conditions of the chromatographic process. For example, if the molecule that is being purified is eluted at a high change in salt concentration, then a ligand and coupling chemistry that is salt tolerant should be selected in order to maintain the reusability of the media. Reactive functional groups that can couple to amine-containing molecules are preferred due to the fact that nearly all protein or peptide molecules are able to be immobilized via an amine-coupling process. Examples of common functional groups used in amine-coupling processes are shown in FIG. 1.

Amine-reactive functional groups include, but are not limited to, isothiocyanates, isocyanates, acyl azides, NHS esters, sulfo-NHS esters, acid chlorides such as sulfonyl chloride, aldehydes and glyoxals, epoxides and oxiranes, carbonates, carbamates, aryl halides, alkyl halides, imidoesters, carbodiimides, anhydrides, fluorophenyl esters, triazines, and combinations thereof. These functional groups can be attached directly to the surface of the membrane support by attachment to the surface of the fibers that make up the membrane, or they can be part of a spacer molecule.

As an example, an epoxide or oxirane group will react with a primary amine nucleophile in a ring-opening process and will result in a secondary amine containing a (3-hydroxy on the epoxy compound anchoring it to the membrane. This process readily occurs at moderate alkaline pH values typically ranging from 8-10. Higher pH values could be used in order to speed up the coupling process as well as increase coupling efficiency under the assumption that the protein or ligand being immobilized is stable under these conditions. The primary side reaction that occurs with the epoxide groups is the hydrolysis of the epoxide ring, which occurs more readily at low pH values, resulting in adjacent hydroxyls. In terms of media such as agarose and cellulose, the hydroxyl groups on the surface of the membrane can be activated with epichlorohydrin at slightly elevated temperatures and highly basic conditions in order to generate functional epoxide groups.

In another example, carboxylic acids can be turned into activated esters for coupling with a nucleophilic primary amine resulting in a secondary amide. Most commonly this activation is used to create an N-hydroxysuccinimide (NHS) ester through either carbodiimides and NHS or disuccinimidyl carbonate (DSC). Then this ester used to immobilize bio-molecules such as enzymes onto supports for purifications and reactions by exchanging the NHS of activated carboxylic acid—NHS ester to form an amide bond with any free nucleophilic amine. This approach is useful because it occurs readily at physiological pH of 8.6 or lower due to much slower NHS hydrolysis and therefor high coupling yields below this pH. NHS coupling is also buffer dependent. The activated esters created prior to coupling can be used immediately or stored under cool anhydrous conditions, so therefor are quite useful for activations. Additional chemistries used in activating functional groups are described in U.S. Pat. No. 8,114,611, which is incorporated herein in its entirety.

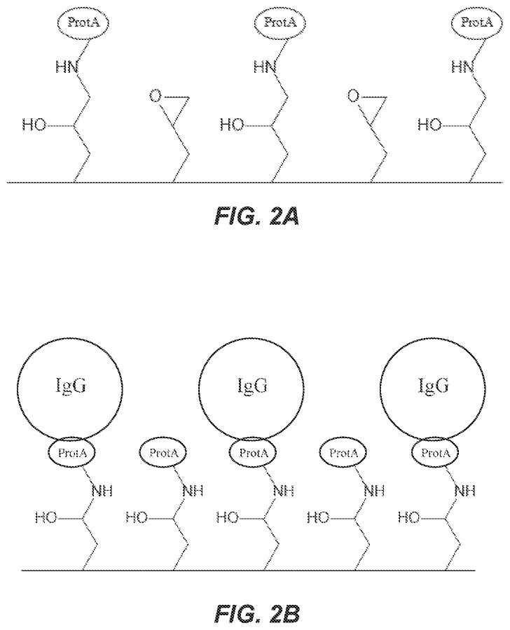

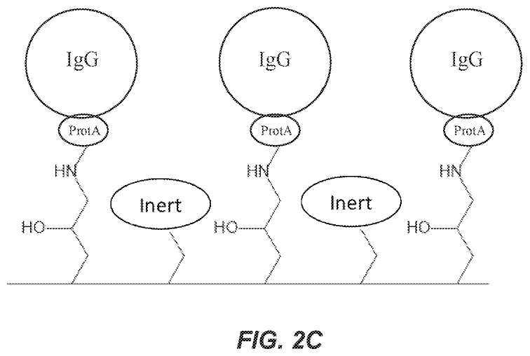

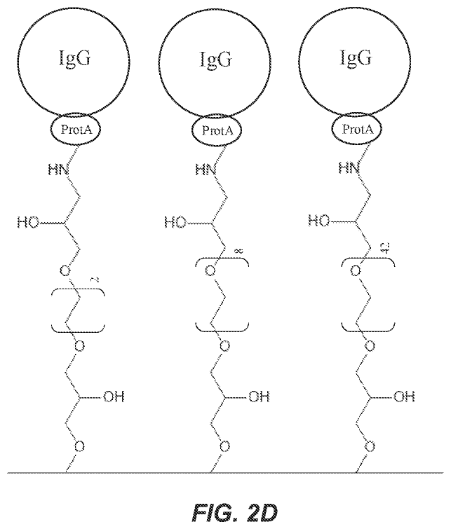

The membrane support surface can be functionalized with reactive functional groups to immobilize affinity ligands and with inert functional groups to improve the efficiency of ligand immobilization and reduce the potential for steric hindrance at the binding sites. A non-limiting example of this is shown FIG. 2C for demonstrative purposes. In this respect the surface of the membrane support is dual functionalized with inert functional groups and binding sites which were reactive functional groups reacted to attach an exemplary affinity ligand, Protein A. The inert functional groups can be selected based on the affinity ligand chosen for a particular application; specifically an inert group can be selected based on it not being reactive with the functionality required to attach the particular affinity ligand. For example, an epoxide ring is reactive with an amine functional group and thus can be a reactive group in certain embodiments; however, in an embodiment where the reactive group is not reactive with an epoxide, then epoxide can be utilized as an inert group.

As described herein, combinations of amine-functional groups can be used to permanently immobilize biomolecular ligands to the surface of the nanofibers of the membrane composition. Additionally, these amine-functional groups can be combined with ion-exchange-functional groups. In one example, some of the hydroxyl groups on the surface of the membrane can be activated with 3-chloro-2-hydroxypropyltrimethyl ammonium chloride (CHPTAC), resulting in the introduction of a quaternary ammonium cation for anion-exchange separations. It is particularly advantageous to use CHPTAC as it results in a net change of zero in regards to hydroxyl groups on the surface. Any residual hydroxyls can then be activated with a bis-oxirane, such as 1,4-butanediol diglycidal ether, resulting in the introduction of an epoxide group for covalent ligand immobilization. The final result is a dual functionalized membrane support having both active coupling functionalities (the affinity ligands) and inert coupling functionalities which prevent steric hindrance at the binding sites and reduce or eliminate inefficient use of immobilized ligands. This dual functionalized membrane support can comprise a quaternary ammonium ion used as a strong attractive center to bring the biomolecular ligand within close proximity of the epoxide group so it can be covalently immobilized.

In another example, some of the hydroxyl groups on the surface of the membrane can be activated with 3-chloro-2-hydroxypropanesulfonic acid sodium salt (CHPSAS), resulting in the introduction of sulfonic acid group for cation-exchange separations. Similarly, as before, the result is a net change of zero in regards to hydroxyl groups on the surface. Any residual hydroxyls can then be activated with a bis-oxirane, such as 1,4-butanediol diglycidal ether, resulting in the introduction of an epoxide group for covalent ligand immobilization. The final result is a dual functionalized material, with a sulfonic acid group that is used a strong attractive center to bring the biomolecular ligand within close proximity of the epoxide group so it can be covalently immobilized. As such, any ligand that can be influenced by an anion or cation attractive center, could potentially benefit from being immobilized with a dual functionalized surface. This dual functionalization can extend to any amine-functional, cation, or anion groups, and combinations thereof An advantage to this is that the ligand density and spacing amongst ligands can be more specifically controlled and tailored.

### Activating Agent Density

Depending on the type of chemistry applied to the nanofiber membrane, an activation density can be determined. Activation density refers to the amount of activating agent covalently bonded per amount of membrane material. For example, in the case of activating the membrane with epoxide groups, the activation density could be represented as the moles of epoxide groups per grams of membrane material. Determining the activation density is an important parameter because it gives an indication to the maximum achievable binding capacity of the material. After the chemistry is applied, and the material is therefore activated, the theoretical binding capacity can never exceed the activation density. Due to steric hinderance and molecular interactions, an efficiency loss will always occur in between activation and coupling, as well as in between coupling and static/dynamic binding. As shown in FIG. 2A, a simplified example demonstrates this loss in site density during the ligand coupling step. Because of these complex molecular interactions, it may be desirable to either lower or raise the activation density in order to achieve an overall higher binding capacity of the nanofiber membranes.

### Coupled Ligand Density

Another parameter to consider is the amount, or rather density, of ligand that is immobilized on the surface of the media. As shown in FIG. 2B, a simplified example demonstrating the effect of ligand concentration on IgG binding is shown under the assumption that only one IgG molecule binds per one Protein A (ProtA) molecule. It should be known that contrary to this example, there is theoretically four potential binding sites for IgG per molecule of immobilized Protein A. However, the effective number of binding sites could be less than four due to the fact that steric and repulsive interactions could prevent the approach, and therefore affinity binding, of IgG as a result of having an overcrowded surface. Nevertheless, FIG. 2B shows that despite there being five Protein A molecules, and thus five potential binding sites in this example, only three IgG molecules are capable of binding to the surface. This results in a loss of Protein A utilization on the surface of the media and therefore higher costs due to the relatively expensive price of Protein A. The efficiency of Protein A utilization can therefore be optimized by changing the ligand density on the surface of the media while also maintaining the IgG binding capacity.

A benefit of the present disclosure is that it provides mechanisms for improving the efficiency by tailoring the surface functionalizations to avoid steric hindrance and repulsive interactions. There are two mechanisms for achieving this. First, the membrane support can be surface functionalized with dual functionalities as described earlier, i.e., by attachment of active binding functionalizations (which bind and immobilize affinity ligands) and inert functionalities to avoid steric hindrance and unnecessary ligand binding. Second, the surface functionalizations can be tailored to include spacer arms of determined lengths to avoid steric hindrance. These two mechanisms can be employed together on the same membrane support. Advantageously, either or both of these mechanisms can be utilized to increase the efficiency of ligand binding by tailoring the surface functionalizations to reduce the chances of steric hindrance and avoid binding of ligands which would go unused because of steric hindrance.

The ligand may be present at density of from about 10 mg/g to about 1000 mg/g. Embodiments provide the ligand may be present at density of from about 10 mg/g to about 100 mg/g, from about 10 mg/g to about 200 mg/g, from about 10 mg/g to about 250 mg/g, from about 10 mg/g to about 300 mg/g, from about 10 mg/g to about 400 mg/g, from about 10 mg/g to about 500 mg/g, from about 10 mg/g to about 600 mg/g, from about 10 mg/g to about 700 mg/g, from about 10 mg/g to about 750 mg/g, from about 10 mg/g to about 800 mg/g, from about 10 mg/g to about 900 mg/g, from about 50 mg/g to about 100 mg/g, from about 50 mg/g to about 200 mg/g, from about 50 mg/g to about 250 mg/g, from about 50 mg/g to about 300 mg/g, from about 50 mg/g to about 400 mg/g, from about 50 mg/g to about 500 mg/g, from about 50 mg/g to about 600 mg/g, from about 50 mg/g to about 700 mg/g, from about 50 mg/g to about 750 mg/g, from about 50 mg/g to about 800 mg/g, from about 50 mg/g to about 900 mg/g, from about 50 mg/g to about 1000 mg/g, from about 100 mg/g to about 200 mg/g, from about 100 mg/g to about 250 mg/g, from about 100 mg/g to about 300 mg/g, from about 100 mg/g to about 400 mg/g, from about 100 mg/g to about 500 mg/g, from about 100 mg/g to about 600 mg/g, from about 100 mg/g to about 700 mg/g, from about 100 mg/g to about 750 mg/g, from about 100 mg/g to about 800 mg/g, from about 100 mg/g to about 900 mg/g, from about 100 mg/g to about 1000 mg/g, from about 200 mg/g to about 250 mg/g, from about 200 mg/g to about 300 mg/g, from about 200 mg/g to about 400 mg/g, from about 200 mg/g to about 500 mg/g, from about 200 mg/g to about 600 mg/g, from about 200 mg/g to about 700 mg/g, from about 200 mg/g to about 750 mg/g, from about 200 mg/g to about 800 mg/g, from about 200 mg/g to about 900 mg/g, from about 200 mg/g to about 1000 mg/g, from about 250 mg/g to about 300 mg/g, from about 250 mg/g to about 400 mg/g, from about 250 mg/g to about 500 mg/g, from about 250 mg/g to about 600 mg/g, from about 250 mg/g to about 700 mg/g, from about 250 mg/g to about 750 mg/g, from about 250 mg/g to about 800 mg/g, from about 250 mg/g to about 900 mg/g, from about 250 mg/g to about 1000 mg/g, from about 300 mg/g to about 400 mg/g, from about 300 mg/g to about 500 mg/g, from about 300 mg/g to about 600 mg/g, from about 300 mg/g to about 700 mg/g, from about 300 mg/g to about 750 mg/g, from about 300 mg/g to about 800 mg/g, from about 300 mg/g to about 900 mg/g, from about 300 mg/g to about 1000 mg/g, from about 400 mg/g to about 500 mg/g, from about 400 mg/g to about 600 mg/g, from about 400 mg/g to about 700 mg/g, from about 400 mg/g to about 750 mg/g, from about 400 mg/g to about 800 mg/g, from about 400 mg/g to about 900 mg/g, from about 400 mg/g to about 1000 mg/g, from about 500 mg/g to about 600 mg/g, from about 500 mg/g to about 700 mg/g, from about 500 mg/g to about 750 mg/g, from about 500 mg/g to about 800 mg/g, from about 500 mg/g to about 900 mg/g, from about 500 mg/g to about 1000 mg/g, from about 600 mg/g to about 700 mg/g, from about 600 mg/g to about 750 mg/g, from about 600 mg/g to about 800 mg/g, from about 600 mg/g to about 900 mg/g, from about 600 mg/g to about 1000 mg/g, from about 700 mg/g to about 750 mg/g, from about 700 mg/g to about 800 mg/g, from about 700 mg/g to about 900 mg/g, from about 700 mg/g to about 1000 mg/g, from about 750 mg/g to about 800 mg/g, from about 750 mg/g to about 900 mg/g, from about 750 mg/g to about 1000 mg/g, from about 800 mg/g to about 900 mg/g, from about 800 mg/g to about 1000 mg/g, or from about 900 mg/g to about 1000 mg/g. Embodiments provide the ligand may be present at density of from about 10 mg/g, about 50 mg/g, about 100 mg/g, about 200 mg/g, about 250 mg/g, about 300 mg/g, about 400 mg/g, about 500 mg/g, about 600 mg/g, about 700 mg/g, about 750 mg/g, about 800 mg/g, about 900 mg/g, or about 1000 mg/g.

### Spacer Molecules (Spacer Arms)

One option to increase ligand immobilization is by introducing a spacer molecule between the nanofiber membrane and the affinity ligand. By introducing a plurality of spacer molecules onto the surface of the membrane support the steric hinderance can be reduced or even eliminated between the matrix and the immobilized ligand as well as the adsorbing species. For example, both Protein A and IgG are very large biomolecules and therefore can have a significant amount of steric hinderance limiting their ability to approach the matrix during both Protein A immobilization and IgG dynamic binding. Additionally, a high ligand density can limit adsorption density due to the steric hinderance blocking potential active sites on ligand molecules within close proximity to one another. Introduction of a long spacer molecule can also indirectly lower ligand density. Decreasing steric hinderance from both the ligand and matrix can allow for an easier approach of the IgG molecule during dynamic binding resulting in increased adsorption capacity.

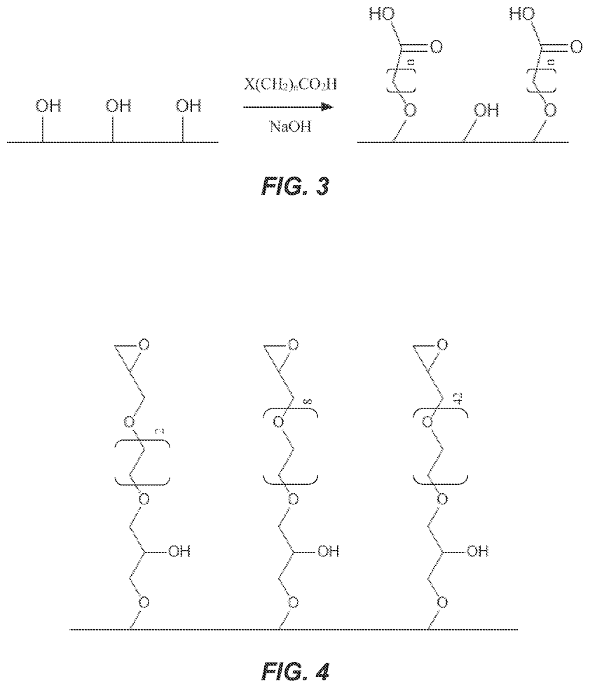

The chemistry of the spacer arm can have large effects on affinity chromatography, depending upon support, immobilized ligand and binding target. This effect can be multi-faceted, owing to not only hydrophobic and hydrophilic interactions, but specific associative bonding from other side chains. An example of introducing a hydrocarbon spacer molecule for subsequent activation with a carboxylic acid is shown is FIG. 3. Incorporation of spacer molecules of hydrophilic nature can prevent non-specific hydrophobic side chain bonding between support, spacer, ligand, and target that promotes either positive or negative bonding influences on the membrane. Hydrophilic effects can be incorporated into a spacer arm through incorporation of electronegative atoms into the spacer molecule as seen with the PEG example (FIG. 4). To maximize affinity vs hydrophobic or hydrophilic binding effects, buffer ionic concentration should be evaluated to maximize affinity binding between protein A and IgG.

Each spacer arm has a terminus where either a reactive functional group or an inert functional group is located. The reactive functional group can be reacted to bind an affinity ligand. In this respect, an affinity ligand can be located at the terminus. The spacer arm is typically a linear chain of carbon, oxygen, and nitrogen atoms. The spacer arm may be comprised of a branched chain, an unbranched chain or a cyclic chain. The compositions may comprise a hydrophilic spacer arm, hydrophobic spacer arm, or a combination of hydrophilic and hydrophobic spacer arms. Preferably the spacer arm has a length of between 1 and 100 atoms, between 1 and 90, between 1 and 80, between 1 and 70, between 1 and 60, between 1 and 50. Embodiments provide the spacer arm has a length of between 1 and 5 atoms, between 1 and 10 atoms, between 1 and 15 atoms, between 1 and 20 atoms, between 1 and 25 atoms, between 5 and 10 atoms, between 5 and 15 atoms, between 5 and 20 atoms, between 5 and 25 atoms, between 5 and 30 atoms, between 10 and 15 atoms, between 10 and 20 atoms, between 10 and 25 atoms, between 10 and 30 atoms, between 15 and 20 atoms, between 15 and 25 atoms, between 15 and 30 atoms, between 20 and 25 atoms, between 20 and 30 atoms, or between 25 and 30 atoms. Embodiments provide the spacer arm has a length of 1 atom, 2 atoms, 3 atoms, 4 atoms, 5 atoms, 6 atoms, 7 atoms, 8 atoms, 9 atoms, 10 atoms, 11 atoms, 12 atoms, 13 atoms, 14 atoms, 15 atoms, 16 atoms, 17 atoms, 18 atoms, 19 atoms, 20 atoms, 21 atoms, 22 atoms, 23 atoms, 24 atoms, 25 atoms, 26 atoms, 27 atoms, 28 atoms, 29 atoms, or 30 atoms.

### Tailoring of all Parameters to Particular Ligand Applications

Coupling chemistries, ligand density, spacer molecule types, and spacer molecule lengths can all be combined and optimized simultaneously to maximize ligand utilization efficiency as well as the dynamic binding capacity of the target of interest during affinity chromatography. Each one of these parameters independently has an impact on the process performance, and when combined leads to overall greater utilization and performance of the adsorption media.

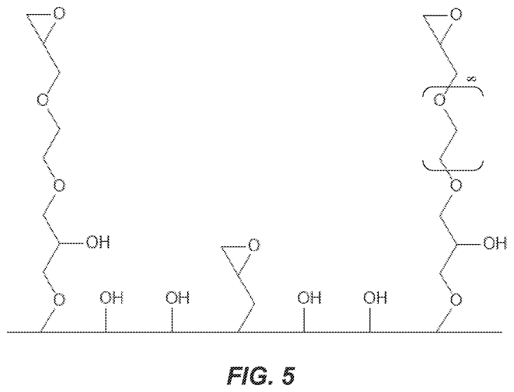

A surface with multiple different spacer molecule lengths could have a greater dynamic binding capacity for target of interest than a surface with only one type of spacer length because spacing out the ligands could lead to a greater availability of the active sites for binding the ligand. An example of this concept is illustrated in FIG. 5 utilizing Protein A as the exemplary ligand.

FIG. 5 demonstrates one possible surface combination with varied ligand density and multiple different spacer molecule lengths, as well as having some hydroxyl groups activated with no spacer molecule. Note that this is only one of many possible combinations that could be created in an attempt to maximize the dynamic binding of IgG and Protein A efficiency. For example, multiple different chemistries, such as NHS esters and epoxide groups, could be used in parallel when activating the membrane. Alternatively, there could be only one type of activating chemistry, but with multiple different linker lengths and types. An example of this would be activating the hydroxyl groups on the cellulose matrix with both hydrophobic and hydrophilic bis-epoxide spacer molecules, along with each having varying spacer arm lengths. For each one of these scenarios, there is also an optimal ligand density that can be determined in order to further optimize performance.

### Methods of Using the Membranes

The surface functionalized affinity membranes can be used to isolate a target of interest from a sample by contacting the sample comprising the target of interest with the composition to adsorb the target of interest. The methods can further comprise contacting the composition with an eluent to release the target of interest and recovering the target of interest. Targets of interest may include any molecule which specifically binds to the ligand of choice. The target molecule may be a protein or a nucleic acid molecule, including a vector, a viral vector, or a virus. In some embodiments, the target molecule is an immunoglobulin such as immunoglobulin G (IgG). Targets of interest may also include any nanoparticle which specifically binds to the ligand of choice. Target nanoparticles may include an encapsulated nanoparticle, a lipid nanoparticle, a virus-like particle, and/or an exosome.

Nucleic acids can be in the form of deoxyribonucleic acid (DNA), ribonucleic acid (RNA) or peptide nucleic acid (PNA), as well as analogs, derivatives, or any combination thereof. Such a derivative could contain, for example, a nucleotide analog or a “backbone” bond other than a phosphodiester bond, for example, a phosphotriester bond, a phosphoranaidate bond, a phosphorodaioate bond, or a thioester bond. Naturally-occurring RNA molecules include, but are not limited to, transfer RNA (tRNA), ribosomal RNA (rRNA), messenger RNA (mRNA), or genomic RNA, such as that from influenza or hepatitis C viruses. Other forms of RNA include, but are not limited to, small interfering RNA (sib NA) and microRNA (miRNA). Thu nucleic acid molecules can be single-stranded (ss; and which can be sense or antisense), double-stranded (ds) or a combination of the two, and can he linear or circular, the latter of which can be open-circular or closed-circular.

The nucleic acid molecule can be a vector. The vector can be a viral vector, preferably a lentivirus vector, an adenovirus vector, an adeno-associated virus (AAV) vector, a vesicular stomatitis virus (VSV) vector, a herpes simplex virus (HSV) vector, a vaccinia virus vector, a pox virus vector, an influenza virus vector, a respiratory syncytial virus vector, a parainfluenza virus vector, a foamy virus vector, or a retrovirus vector.

In an aspect of the invention, the compositions can provide an enhanced binding capacity, e.g., static capacity and dynamic capacity, of the target molecule as compared to previously described methods. The compositions can have a static binding capacity on a mass basis of between about 10 mg/g and about 200 mg/g, preferably between about 60 mg/g and about 100 mg/g.

### EXAMPLES

Preferred embodiments of the present disclosure are further exemplified in the following non-limiting Examples. It should be understood that these Examples, while indicating certain preferred embodiments, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

Ligand-functionalized membranes were produced according to the methods described herein and evaluated for various factors. These factors included Protein A coupling, dynamic binding and static binding. Coupling was evaluated by either BCA or Bradford assays, where the original concentration was compared to remaining concentration to determine the amount of ligand bound.

To evaluate performance, a GE Akta Pure 150 was used. Equilibration buffer used was 0.1M Sodium Phosphate pH 7.0, 1 mg/ml Human IgG in 0.1M Sodium Phosphate pH 7.0 used for binding, and 0.1M Glycine pH 2.5 used for elution. To calculate amount IgG bound, elution peak integration was taken and total amount bound calculated. Static capacity was measured by shaking sample in known solution of 1 mg/ml Human IgG in 0.1M Sodium Phosphate pH 7.0 for 24 hours, proceeding with Bradford IgG remaining against knows standard.

### Example 1: Epoxide Functionalities

Epoxide Coupling without Spacer Molecule, Epichlorohydrin and Protein A

Hybrid cellulose nanofiber membranes were epoxide activated using Epichlorohydrin according to the following method: 15 mL of 1M NaOH was preheated to 60° C. in a hot water bath. The hybrid cellulose nanofiber membrane material (0.0255 g) was soaked in the previous solution, and placed on an air-heated rotary shaker at a temperature of 60° C. After 15 minutes of soaking, 1.5 mL of Epichlorohydrin (Epi.) was added and the reaction shaken vigorously (>100 rpm) for 4 hours at a temperature of 60° C. The membrane material was then washed with reverse osmosis (RO) H₂O for an ample amount of time (>30 min). The maximum potential epoxide groups were estimated based on the amount of activating agent that was used.

The epoxide activated cellulose nanofiber membrane material was coupled with Protein A according to the following method: A coupling buffer was prepared and comprised of 1M Ammonium Sulfate and 0.1M Sodium Carbonate with a pH of 8.75 adjusted using HCl. The membrane material was added to 900 μL of coupling buffer and 100 μL of 50 mg/mL Protein A (Repligen). The membrane was shaken (>100 rpm) for 24 hours at room temperature. After coupling, unreacted epoxide groups were blocked using a 1M Ethanolamine solution pH adjusted to 9 using HCl. The final amount of Protein A in the solution was determined using a Pierce BCA Protein Assay Kit (Thermo Fischer Scientific) with Protein A as the standard curve. The maximum amount of Protein A coupled to the membrane material was taken to be the difference between the initial protein concentration added and the final protein concentration.

Epoxide Coupling with Spacer Molecule, Poly(ethylene glycol) Diglycidyl Ether (Average Mₙ=500) and Protein A

Hybrid cellulose nanofiber membranes were epoxide activated using Epichlorohydrin according to the following method: 15 mL of 1M NaOH was prepared. The hybrid cellulose nanofiber membrane material (0.0264 g) was soaked in the previous solution, and placed on an rotary shaker at room temperature. After 15 minutes of soaking, 187.5 μL of Poly(ethylene glycol) diglycidyl ether, average molecular weight of 500, (PEG-500) was added and the reaction shaken vigorously (>100 rpm) for 4 hours at room temperature. The membrane material was then washed with RO H₂O for an ample amount of time (>30 min). The maximum potential epoxide groups were estimated based on the amount of activating agent that was used.

The epoxide activated cellulose nanofiber membrane material was coupled with Protein A according to the following method: A coupling buffer was prepared and comprised of 1M Ammonium Sulfate and 0.1M Sodium Carbonate with a pH of 8.75 adjusted using HCl. The membrane material was added to 900 μL of coupling buffer and 100 μL of 50 mg/mL Protein A (Repligen). The membrane was shaken (>100 rpm) for 24 hours at room temperature. After coupling, unreacted epoxide groups were blocked using a 1M Ethanolamine solution pH adjusted to 9 using HCl. The final amount of Protein A in the solution was determined using a Pierce BCA Protein Assay Kit (Thermo Fischer Scientific) with Protein A as the standard curve. The maximum amount of Protein A coupled to the membrane material was taken to be the difference between the initial protein concentration added and the final protein concentration. Table 1 shows a summary of epoxide-Protein A coupling results.

Epoxide Coupling with Spacer Molecule, Poly(ethylene glycol) Diglycidyl Ether (Average Mₙ=2000) and Protein A

Hybrid cellulose nanofiber membranes were epoxide activated using Epichlorohydrin according to the following method: 0.1681 g of Poly(ethylene glycol) diglycidyl ether, average molecular weight of 2000, (PEG-2000) was fully dissolved in 15 mL of 1M NaOH. The hybrid cellulose nanofiber membrane material (0.0268 g) was soaked in the previous solution, and placed on a rotary shaker and the reaction shaken vigorously (>100 rpm) for 4 hours at room temperature. The membrane material was then washed with RO H₂O for an ample amount of time (>30 min). The maximum potential epoxide groups were estimated based on the amount of activating agent that was used.

The epoxide activated cellulose nanofiber membrane material was coupled with Protein A according to the following method: A coupling buffer was prepared and comprised of 1M Ammonium Sulfate and 0.1M Sodium Carbonate with a pH of 8.75 adjusted using HCl. The membrane material was added to 900 μL of coupling buffer and 100 μL of 50 mg/mL Protein A (Repligen). The membrane was shaken (>100 rpm) for 24 hours at room temperature. After coupling, unreacted epoxide groups were blocked using a 1M Ethanolamine solution pH adjusted to 9 using HCl. The final amount of Protein A in the solution was determined using a Pierce BCA Protein Assay Kit (Thermo Fischer Scientific) with Protein A as the standard curve. The maximum amount of Protein A coupled to the membrane material was taken to be the difference between the initial protein concentration added and the final protein concentration. Table 1 shows a summary of epoxide-Protein A coupling results.

TABLE 1

| | Activation | | Protein A | |
|---|---|---|---|---|
| | (g Epoxide | Protein A | Density | Protein A |
| Protein A | added/g | Estimation | (mg/g | Coupled |
| Sample | membrane) | (mg) | membrane) | (%) |
| Epi. | 69.59 | 4.564 | 179.0 | 91.28 |
| PEG-500 | 8.10 | 4.673 | 177.0 | 93.45 |
| PEG-2000 | 6.27 | 4.673 | 174.4 | 93.45 |

The IgG static binding capacity of the Protein A coupled cellulose nanofiber membrane materials were determined according to the following method: A solution of 1 mg/mL of IgG in 0.1M NaHPO₄ was prepared at pH 7. Protein A coupled cellulose nanofibers were placed in 2 mL of the previously mentioned solution and bound overnight on a rotary shaker (>100rpm). Using IgG as a standard curve, IgG in the solution was determined using a Bradford Protein Assay (SigmaAldrich). The maximum amount of IgG bound to the membrane material was taken to be the difference between the initial IgG concentration added and the final IgG concentration. Dynamic binding performance was determined from the previously mentioned methods above. A summary of the results for both the static and dynamic binding of IgG to the epoxide activated, Protein A coupled cellulose nanofiber membranes is shown below in Table 2.

TABLE 2

| | Sample | | Max UV | Peak | Peak Area | |
|---|---|---|---|---|---|---|
| | Volume | Q_{static} | (mAU · | Height | (mAU · | Q_{dynamic} |
| Sample | (mL) | (mg/g) | mL/mg) | (mAU) | mL) | (mg/g) |
| Epi. | 0.03255 | 6.46 | 276.1 | 13.67 | 18.29 | 2.60 |
| PEG- | 0.02418 | 14.43 | 267.5 | 26.40 | 26.40 | 3.74 |
| 500 | | | | | | |
| PEG- | 0.02697 | 15.81 | 265.2 | 27.88 | 30.48 | 4.29 |
| 2000 | | | | | | |

The results shown in Table 1 demonstrate the effect that functional group density and spacer arm length has on the amount of Protein A that can be immobilized onto the matrix. The first row indicates an epoxide group with no active binding site, the row employs a PEG-500 as a spacer arm, and the third employs a PEG-2000 as a longer spacer arm. Experimentally, the maximum potential moles of epoxide groups that were added to the surface decreased with increasing spacer length, thereby simultaneously varying two parameters. Despite the fact that there were fewer potential moles of epoxide for the Protein A to couple to, the amount of Protein A coupled to the membrane increased with increasing spacer arm length.

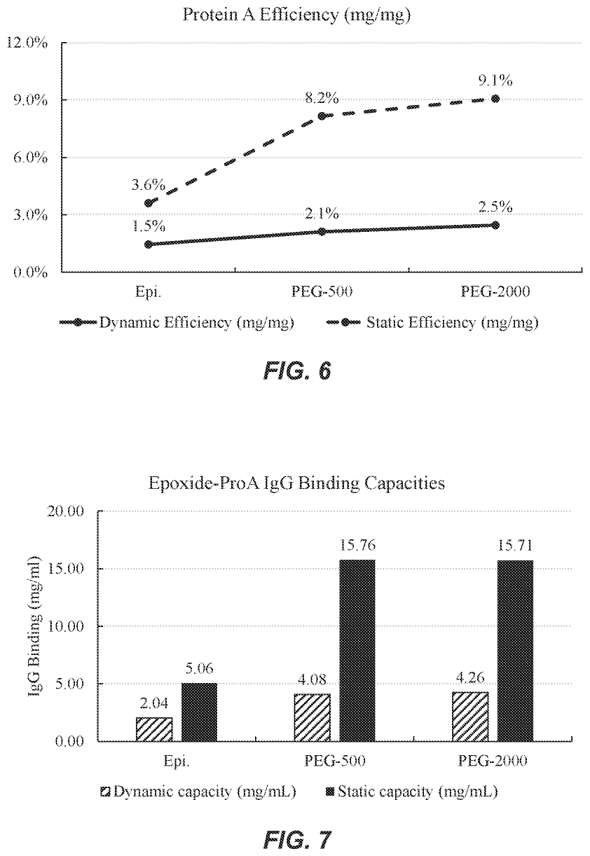

FIG. 6 demonstrates the effect that varying spacer molecule length has on the efficiency of the immobilized Protein A. For both static and dynamic binding results show that an increase in the length of the spacer molecule results in a greater utilization of the Protein A.

Table 2 and FIG. 7 demonstrate the effect that increasing spacer molecule length has on the static and dynamic binding capacity of IgG. Results showed that introducing a spacer molecule increased the dynamic binding from 2.60 mg/mL to 3.74 mg/mL, and that increasing the length of the spacer molecule increased the dynamic binding from 3.74 mg/mL to 4.29 mg/mL. These results are supported by the fact that the elution peak height and integrated area correspondingly increase with increasing spacer molecule length. Similarly, for static binding, introducing a spacer molecule increased binding from 6.46 mg/mL to 14.43 mg/mL, and increasing the length of the spacer molecule increased binding from 14.43 mg/mL to 15.81 mg/mL.

### Example 2: NHS Functionalities

To explore the conjugation with nanofiber membranes, cellulose nanofibers were reacted with various length halo-alkanoic acids in a caustic solution. In this case, the halo-alkanoic acids included monochloroacetic acid (MCA), bromohexanoic acid (BrHex), bromoundecanoic acid (uBr), and a combination of one third of each of those reacted onto the surface of the nanofibers. The membranes were pre-soaked for 15 minutes in 60° C. 1.2M NaOH at 0.4 L/g membrane followed by the addition of alkanoic acid at 0.7 mol/L solution with vigorous shaking to dissolve. This reaction was heated under shaking at 60° C. for 4 additional hours, washed of reactants and stored on RO water. This resulted in the grafting of various alkanoic acids through an ether bond onto the cellulose nanofibers at different lengths. The resulting membrane was characterized for capacity and flow conditions based on lysozyme cation-exchange (CEX) capacity at pH 7 in 20 mM Sodium Phosphate. Further, these were coupled with protein A to determine the effect that varying spacer arm length would have on IgG retention capabilities.

The CEX membranes were then activated using disuccinimidyl carbonate (DSC), triethylamine (TEA), and acetonitrile. First membranes were washed with 0.3M HCl, rinsed repeated with RO water, and subsequently dehydrated using 25:75, 50:50, 75:25 acetone/water mixtures followed by two 15-minute washes of pure to ensure complete dehydration. Thereafter the membranes were added to a mixture of 167 ml anhydrous acetonitrile and 83.5 ml TEA per gram membrane (dry basis) and activated with 54 g DSC/L acetonitrile under shaking. This activation was run for 12 hours. Membranes were then washed clean and stored on isopropyl alcohol (IPA) in a refrigerator at 4° C. To check activation, a coin was removed from the IPA, rinsed with ice cold 1 mM HCl and shaken in 20 mM tris buffer pH 9. UV 280 nm was used to estimate the amount of NHS bound per gram of membrane material.

To couple, a 0.1M sodium bicarbonate 0.5M NaCl buffer salt was mixed into Repligen rSPA Protein A to produce a pH 7.8 solution of 50 mg/ml Protein A. Small test coins of activated membranes were washed free of IPA with ice cold 1mM HCl water before being placed in 33.3 ml Protein A solution/gram membrane with about 0.01 grams membrane used per sample and 3 samples used per type of membrane. The membranes were shaken for 12 hours at 4° C. After, membranes were removed from solution, washed with water. This was followed by shaking with a 20 mM Tris 1.0M NaCl pH 9 for 4 hours to check for adsorbed Protein A.

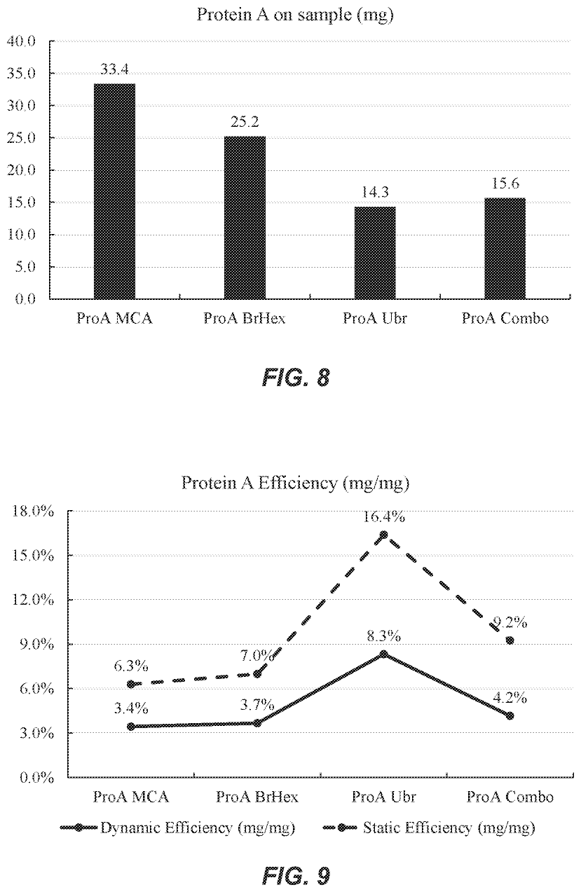

Table 3 shows the summary of NHS-Protein A coupling results. The results indicate that for an increased linker length, the amount of Protein A attached through NHS chemistry decreases, despite an increase in activation of the membrane (the MCA being the shortest spacer arm length and the Ubr being the longest spacer arm length). Note the there is a slight increase in the attached amount of Protein A in the combined sample due to having a proportion of short extenders. This effect is further exemplified in FIG. 8. One would expect this to have a detrimental effect on the ending IgG binding performance.

TABLE 3

| | | | Protein A | |
|---|---|---|---|---|
| | Activation | Protein A | Density | |
| Protein A | (mol NHS/g | Estimation | (mg/g | % ProA |
| Sample | membrane) | (mg) | membrane) | Absorbed |
| MCA | 0.063 | 33.4 | 1113.3 | 66.71% |
| BrHex | 0.102 | 25.2 | 840.0 | 50.41% |
| Ubr | 0.086 | 14.3 | 476.7 | 28.57% |
| Combo | 0.065 | 15.6 | 520.0 | 31.28% |

Longer linker length in hydrophobic spacer arm can also be shown to influence how effective Protein A is at binding to IgG. FIG. 9 shows how a longer linker length increases the binding efficiency Protein A has toward IgG. In varying the linker length from 2 carbon atoms to 11 carbon atoms, the efficiency (mg/mg) that Protein A has on binding to IgG increases from 3.4% to 8.3% for dynamic binding and from 6.3% to 16.4% for static binding. This could be attributed to any number of factors including hydrophobic chemistry of spacer arm, decreased density of binding site, or reduction of protein A-IgG binding hinderance due to increased linker length placing binding further from support matrix.

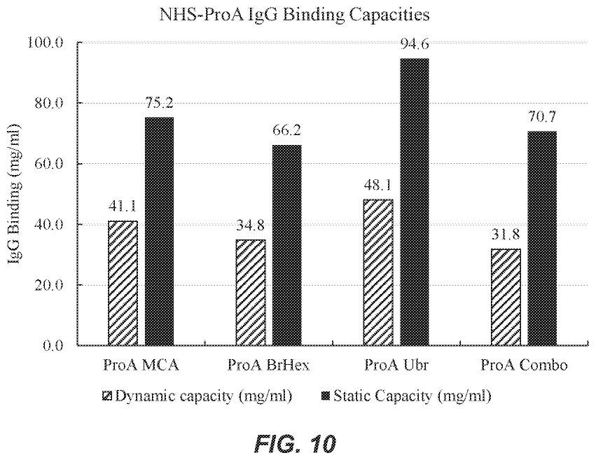

Table 4 shows a summary of static and dynamic IgG binding on NHS-Protein A coupled membranes. Table 4 and FIG. 10 demonstrate the effect that increasing length can have on binding capacities. Note that the lowest Protein A loading with longest linker has the highest capacity, with 78 mg/g static and 40 mg/g dynamic capacity. This could be due to both ligand density effect and length of spacer.

TABLE 4

| | Sample | | Max UV | Peak | Peak Area | |
|---|---|---|---|---|---|---|
| | Volume | Q_{static} | (mAU · | Height | (mAU · | Q_{dynamic} |
| Sample | (mL) | (mg/g) | mL/mg) | (mAU) | mL) | (mg/g) |
| MCA | 0.0279 | 70.0 | 275 | 365.9 | 315.3 | 38.2 |
| BrHex | 0.026598 | 58.7 | 275 | 254.3 | 254.3 | 30.8 |
| Ubr | 0.024738 | 78.0 | 275 | 465.0 | 327.0 | 39.6 |
| Combo | 0.02046 | 48.2 | 275 | 325.9 | 178.9 | 21.7 |

The foregoing examples, demonstrate that affinity ligands can be immobilized on the surface of a membrane support through surface functionalizations. Moreover, it was demonstrated that the spacer arm length can be tailored to avoid steric hindrance and that dual functionalizations can be utilized on the surface of the membrane support to improve the binding efficiency. By tailoring the surface functionalizations, optimal conditions can be arrived at for a particular affinity ligand and its target of interest to maximize the effective ligand density, the ligand binding efficiency, and the avoidance of steric hindrance. While Protein A was often utilized in the foregoing examples as the affinity ligand, the invention is in no way limited to the use of Protein A; rather the use of Protein A was for consistency amongst the examples and demonstrative purposes only. The teachings provided in this disclosure can be applied to various affinity ligands to separate various targets of interest.

The invention being thus described, it will be obvious that the same may be varied in many ways. Such variations are not to be regarded as a departure from the spirit and scope of the inventions and all such modifications are intended to be included within the scope of the following claims. The above specification provides a description of the manufacture and use of the disclosed compositions and methods. Since many embodiments can be made without departing from the spirit and scope of the invention, the invention resides in the claims.

1. An affinity membrane comprising:
a membrane support; wherein the membrane support is a cast membrane support, an electrospun membrane support, or a combination thereof;
a plurality of linear spacer arms of determined length immobilized on the membrane support; wherein the linear spacer arms each comprise a terminus; and
a ligand coupled to the terminus;
wherein the ligand comprises a peptide, a protein, or both.

2. The membrane of claim 1, wherein the membrane support comprises nanofibers, microfibers or a combination thereof.

3. The membrane of claim 1, wherein the membrane support is an electrospun membrane support.

4. The membrane of claim 1, wherein the membrane support further comprises one or more inert functional groups immobilized on the surface of the membrane support.

5. The membrane of claim 1, wherein the plurality of linear spacer arms comprise linear spacer arms of differing lengths.

6. The membrane of claim 1, wherein the ligand is coupled to the terminus of the linear spacer arm via a an amine-reactive functional group.

7. The membrane of claim 1, wherein the nanofiber membrane comprises cellulose, a non-cellulose polymer, or both cellulose and a non-cellulose polymer.

8. The membrane of claim 4, wherein the one or more inert functional groups comprise an anionic group, a cationic group, a neutral group, or combination thereof.

9-11 (canceled)

12. The membrane of claim 1, wherein the spacer arm has a length of between 1 and 30 atoms.

13. (canceled)

14. The membrane claim 1, wherein the ligand is present on the nanofiber membrane support at density of up to about 1000 mg/g.

15. The membrane of claim 1, wherein the protein is an affimer, an antibody, or an antibody fragment.

16. The membrane of claim 15, wherein the protein is Protein A, Protein G, or Protein L.

17. The membrane of claim 1, wherein the ligand can bind a protein or a nucleic acid molecule.

18. The membrane of claim 1, wherein the ligand can bind at least one of a DNA, an RNA, a vector, a viral vector, a virus, an exosome, or an immunoglobulin.

19. A method of preparing a surface functionalized affinity membrane, the method comprising:
providing a membrane support; wherein the membrane support is a cast membrane support, an electrospun membrane support, or a combination thereof;
immobilizing a plurality of linear spacer arms on the membrane support, wherein the linear spacer arms each comprise a terminus; and
coupling a ligand to the terminus of the linear spacer arm via a reactive functional group; wherein the ligand comprises a peptide or protein.

20. (canceled)

21. The method of claim 19, wherein the providing step comprises electrospinning a membrane support.

22-36 (canceled)

37. A method of isolating a target of interest from a sample comprising:
contacting the sample comprising the target of interest with the affinity membrane of claim 1 to adsorb the target of interest.

38. The method of claim 37 further comprising:
contacting the affinity membrane with an eluent to release the target of interest; and recovering the target of interest.

39. The method of claim 37, wherein the target of interest is a protein or a nucleic acid molecule.

40. The method of claim 37, wherein the target of interest is a DNA, an RNA, a vector, a viral vector, a virus, an exosome, an immunoglobulin, a nanoparticle, an encapsulated nanoparticle, a lipid nanoparticle, or a virus-like particle.

41. The method of claim 40, wherein the immunoglobulin is immunoglobulin G (IgG).

42. (canceled)

43. The method of any one of claim 37, wherein the sample is a biological sample.

44. The method of claim 43, wherein the biological sample is cell culture fluid.

45. An affinity membrane comprising:
(i) an electrospun nanofiber membrane support; and
(ii) a ligand comprising a small-molecule biomimetic ligand, wherein the small-molecule biomimetic ligand is attached to the electrospun nanofiber membrane, optionally via a spacer-arm linkage.

46. The affinity membrane of claim 45, wherein the small-molecule biomimetic ligand is attached to the electrospun nanofiber membrane via a spacer-arm linkage.

47. The affinity membrane of claim 45, wherein the ligand comprises two or more of a hydrophilic group, a hydrophobic group, and an ionic group.

48. The affinity membrane according to claim 45 wherein the electrospun nanofiber membrane comprises cellulose fibers, non-cellulose polymer fibers or a combination of cellulose and non-cellulose polymer fibers.

49. An affinity membrane according to claim 46, wherein the spacer-arm linkage is a linear spacer arm of determined length.

50. An affinity membrane comprising:
(i) an electrospun nanofiber membrane support; and
(ii) a ligand comprising a nucleic acid, an aptamer, or an oligonucleotide, wherein the nucleic acid, aptamer, or oligonucleotide is attached to the electrospun nanofiber membrane, optionally via a spacer-arm linkage.

51. The affinity membrane according to claim 50 wherein the electrospun nanofiber membrane comprises cellulose fibers, non-cellulose polymer fibers or a combination of cellulose and non-cellulose polymer fibers.

52. An affinity membrane according to claim 50, wherein the spacer-arm linkage is a linear spacer arm of determined length.

53. An affinity membrane comprising:
(i) an electrospun nanofiber membrane support and
(ii) a ligand comprising a polysaccharide ligand, an oligosaccharide ligand or a sugar ligand; wherein the ligand is attached to the electrospun nanofiber membrane, optionally via a spacer-arm linkage.

54. The affinity membrane according to claim 53 wherein the electrospun nanofiber membrane comprises cellulose fibers, non-cellulose polymer fibers or a combination of cellulose and non-cellulose polymer fibers.

55. An affinity membrane according to claim 53, wherein the spacer-arm linkage is a linear spacer arm of determined length.

56. An affinity membrane comprising:
(i) an electrospun nanofiber membrane support; and
(ii) a ligand comprising an antigen ligand, an enzyme substrate or an enzyme inhibitor; wherein the ligand is attached to the electrospun nanofiber membrane, optionally via a spacer-arm linkage.

57. The affinity membrane according to claim 56 wherein the electrospun nanofiber membrane comprises cellulose fibers, non-cellulose polymer fibers or a combination of cellulose and non-cellulose polymer fibers.

58. An affinity membrane according to claim 56, wherein the spacer-arm linkage is a linear spacer arm of determined length.
